(19) United States
(12) Patent Application Publication
(10) Pub. No.: US 2025/0064444 A1
(43) Pub. Date: Feb. 27, 2025

(54) SELF-CINCHING SUTURE DEVICES AND METHODS

(71) Applicant: INS Ortho, Inc., Providence, RI (US)

(72) Inventor: Christian N. Anderson, Nashville, TN (US)

(21) Appl. No.: 18/582,622

(22) Filed: Feb. 20, 2024

Related U.S. Application Data
(60) Provisional application No. 63/446,742, filed on Feb. 17, 2023.

(51) Int. Cl. A61B 17/04

(52) U.S. Cl. CPC A61B 17/0401; A61B 2017/0459

(57) ABSTRACT

A self-cinching suture device includes a suture body forming an adjustable loop, and including a self-cinching section wherein the suture body passes through itself in one or more axial or transverse passes. In addition to the self-cinching section, the suture body includes a transverse pass where the suture body passes transversely through itself and makes a 180 degree turn back toward the adjustable loop.

### TECHNICAL FIELD

The present disclosure relates to devices and methods for orthopedic surgery, and more particularly to devices and methods for stabilizing a joint using flexible suture devices and methods to repair and reconstruct ligaments and tissue.

### BACKGROUND

Conventional devices and methods for repair and reconstruction of ligaments and tissue include flexible sutures that may be attached to tissue and anchored to surrounding tissue or bone to place the tissue in an anatomically correct position to facilitate healing and growth. Conventional suture constructs are generally secured to tissue by forming knots in the suture material to maintain tension on the tissue; however, such knots can be difficult to employ in the surgical field, and the knots may loosen over time which leads to slow healing of the tissue.

Further, conventional suture devices for securing free tissue or grafts of tissue during repair or reconstruction of torn or damaged ligaments such as labrum, rotator cuff, or other ligaments and tendons may loosen after cyclical loading of the joint. Additionally, conventional knotted devices have drawbacks in specific procedures where a tendon must be drawn into a hollow anchor, socket, tunnel or near a bone with a limited range of motion. Conventional suture devices have a tendency to bottom out when a travel limit is reached, which may limit the amount of tension that can be applied to the device.

What is needed then, are improvements in self-cinching suture devices and methods of repairing and reconstructing tissue in orthopedic procedures.

### BRIEF SUMMARY

The present disclosure provides self-cinching suture devices and methods of use during orthopedic procedures such as repair or reconstruction of torn or damaged ligaments and tendons.

In some embodiments, the present disclosure provides a self-cinching suture device comprising a suture body forming an adjustable loop, and including a self-cinching section wherein the suture body passes through itself in one or more axial or transverse passes. In addition to the self-cinching section, the suture body includes a transverse pass where the suture body passes transversely through itself and makes a 180 degree turn back toward the adjustable loop. In some embodiments, the suture device includes a fixed loop at one end, and the transverse pass is located between the fixed loop and the self-cinching section. In other embodiments, a fixed anchor is disposed on the end of the suture device instead of the fixed loop, and the transverse pass is located between the fixed anchor and the self-cinching section. In further embodiments, the transverse pass is located adjacent to the self-cinching section and self-cinching sleeve such that the suture strand exits the transverse pass and immediately enters the self-cinching sleeve.

### DESCRIPTION OF THE DRAWINGS

FIGS. 1-26 illustrate various embodiments of self-cinching suture devices and methods for use with associated anchors for orthopedic procedures.

### DETAILED DESCRIPTION

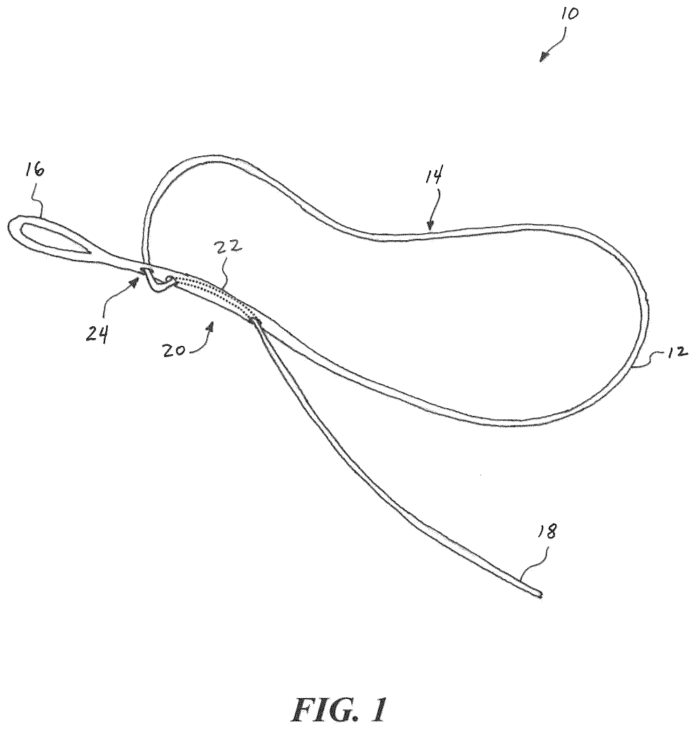

The present disclosure provides various embodiments of improved self-cinching suture devices and methods for repair and reconstruction of ligaments and tendons during orthopedic surgery. Referring to FIG. 1, an embodiment of a self-cinching suture device 10 includes a suture body 12 forming an adjustable loop 14. Suture device 10 includes any suitable suture material including woven or non-woven flexible suture material such as but not limited to hollow-core suture material, braided suture material, suture tape, or any other suitable suture material known in the art. Suture device 10, shown in FIG. 1, includes a single strand of suture material having a fixed loop 16 at a first end, and an adjustable loop 14 is formed by passing the suture body 12 through itself in a transverse pass 24 travelling through the suture material near the fixed loop 16. The tag end 18 then passes through a self-cinching section 20 formed in the suture material. The transverse pass 24 is between fixed loop 16 and self-cinching section 20. Transverse pass 24 includes the suture strand passing transversely through a hole defined in the suture material.

Self-cinching section 20 includes passing the suture strand axially through its own hollow core for a limited distance, as shown in FIG. 1. The suture strand enters the hollow core of the suture material at a first opening, travels axially inside the suture material, and the tag end 18 exits the suture material at a second opening in a direction away from the self-cinching section 20. As such, the self-cinching sleeve 22 functions to compress against the portion of the suture strand housed inside the hollow core during tensioning of the device, thereby locking the suture material against itself inside the self-cinching sleeve 22 when tightened. During use, a user may pull on tag end 18 to tighten the device, and transverse pass 24 provides a fixed location to pull against, thereby drawing the suture strand through the transverse pass 24 and tightening adjustable loop 14.

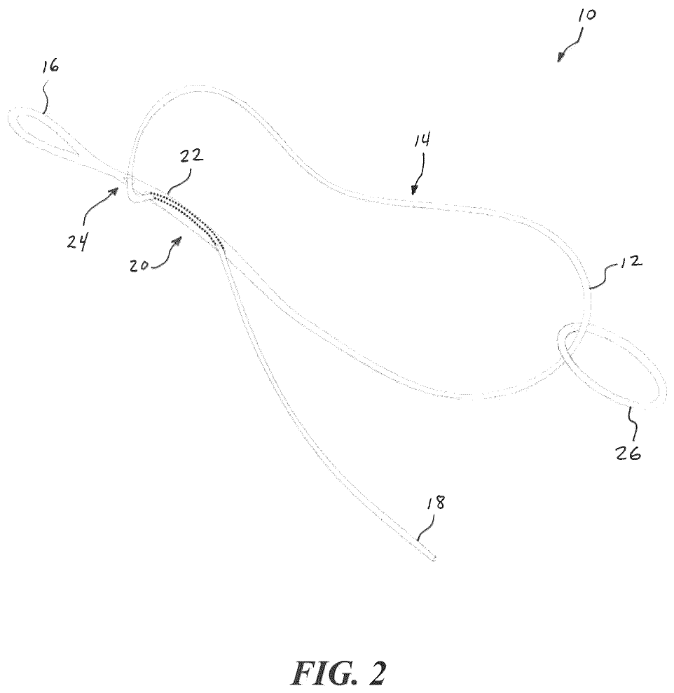

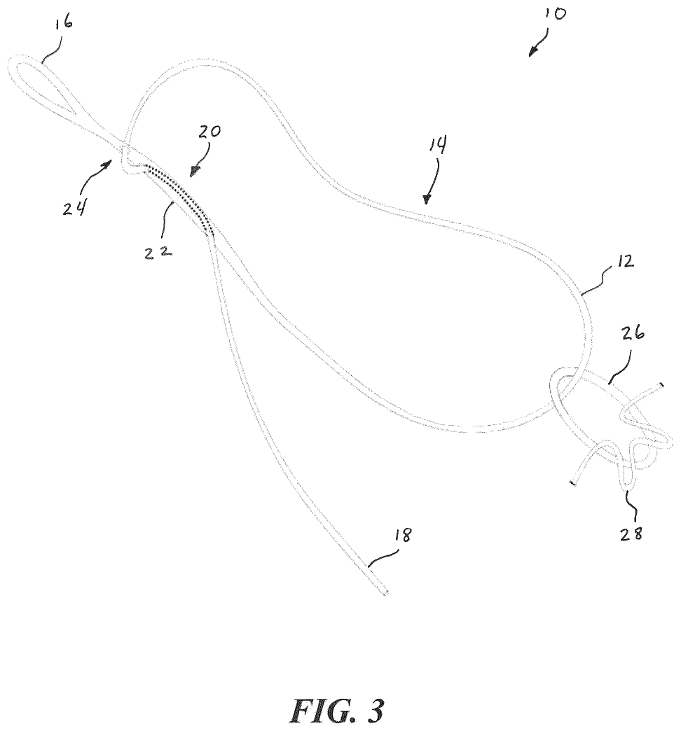

Referring to FIG. 2, an additional embodiment of a self-cinching suture device 10 includes a similar construction as the device depicted in FIG. 1, and further including a free loop 26 disposed on the adjustable loop 14. Free loop 26 provides a location to attach an anchor or another suture opposite fixed loop 16. For example, referring to FIG. 3, an embodiment of a self-cinching suture device 10 includes a first anchor 28 disposed on free loop 26. First anchor 28 can include any suitable anchor known in the art, such as but not limited to flexible suture tape, button, suture material, metal anchor, PEEK anchor, plastic anchor, implant, graft, tissue graft, screw, plate, needle, suture loop, bioresorbable anchor, or other orthopedic devices. As shown in FIG. 3, in some embodiments, first anchor 28 includes a soft anchor comprising flexible suture tape installed on free loop 26 using multiple transverse passes of the suture strand through the anchor material. In some embodiments, the first and second tag ends of first anchor 28 extend radially from free loop 26 facing adjustable loop 14 to provide enhanced engagement with tissue.

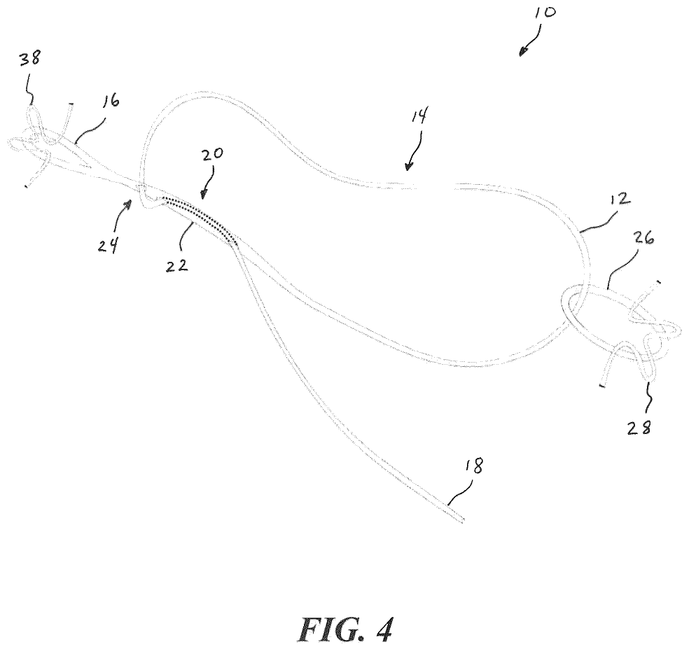

Referring to FIG. 4, in some embodiments, suture device 10 includes a first anchor 28 disposed on free loop 26 and a second anchor 38 disposed on fixed loop 16 on the opposite end of suture device 10. Second anchor 38 can include any suitable anchor known in the art, such as but not limited to flexible suture tape, button, suture material, metal anchor, PEEK anchor, plastic anchor, implant, graft, tissue graft, screw, plate, needle, suture loop, bioresorbable anchor, or other orthopedic devices. In some embodiments, second anchor 38 includes a soft anchor comprising flexible suture tape installed on fixed loop 16 using multiple transverse passes of the suture strand through the anchor material. In some embodiments, the first and second tag ends of second anchor 38 extend radially from fixed loop 16 facing adjustable loop 14 to provide enhanced engagement with tissue.

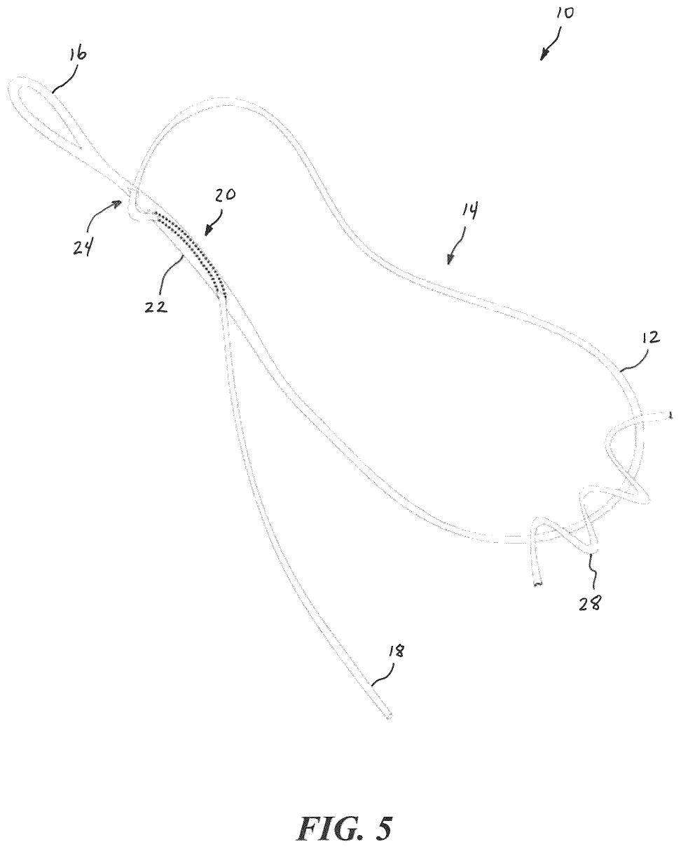

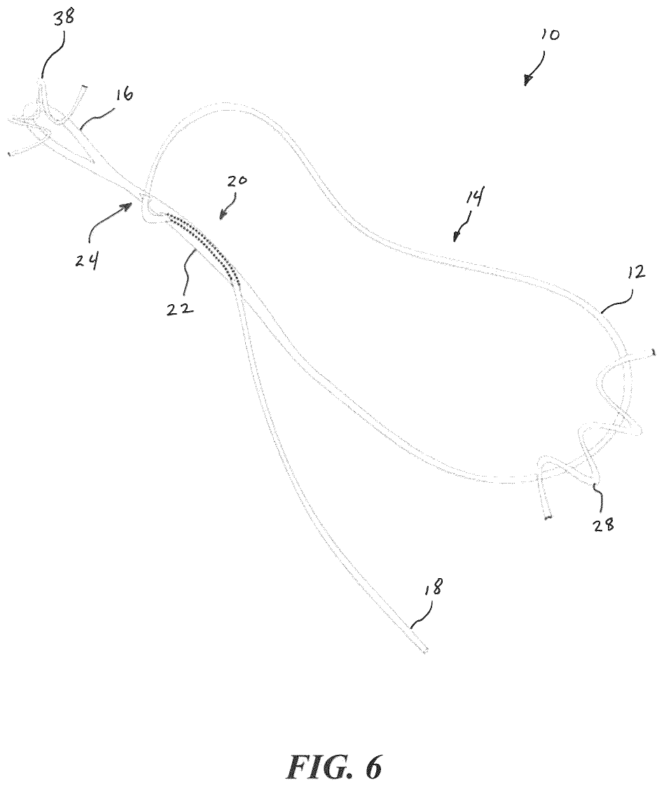

Referring to FIG. 5, in further embodiments, suture device 10 includes a first anchor 28 disposed directly on adjustable loop 14 on the end opposite fixed loop 16. In this embodiment, suture body 12 can slide freely through first anchor 28 as tag end is pulled to tighten adjustable loop 14. Additionally, as shown in FIG. 6, in some embodiments, suture device 10 includes a first anchor 28 disposed directly on adjustable loop 14, and a second anchor 38 disposed on fixed loop 16.

Suture device 10 in some embodiments provides a transverse pass 24 between self-cinching section 20 and fixed loop 16. At the transverse pass 24, the suture strand passes through itself transversely, either by piercing through the suture material or by passing through a pre-defined hole in the suture material sized to accommodate passage of the suture. The suture strand makes a 180-degree turn at the transverse pass 24. When tag end 18 is pulled to tighten adjustable loop 14, the suture material slides through the transverse pass. As such, transverse pass 24 operates as an additional locking mechanism in the suture construct. In some embodiments, the suture device 10 includes a first locking mechanism at first anchor 28 to secure a first end of the device to tissue, bone or an anchor, a second locking mechanism at second anchor 38 to secure a second end of the device to tissue, bone or an anchor, a third locking mechanism at the self-cinching section 20, and a fourth locking mechanism at transverse pass 24. As such, transverse pass 24 contributes to securing the adjustable loop at a desired tension, operating in cooperation with the other locking mechanisms, by providing a 180-degree turn through the suture material itself which provides additional friction on the adjustable loop 14 when tightened. All four locking mechanisms operate together to maintain tension on the suture device 10 when installed in a patient at a desired tension. Transverse pass 24 further prevents loosening of the adjustable loop 14 during cyclic loading of the joint over time.

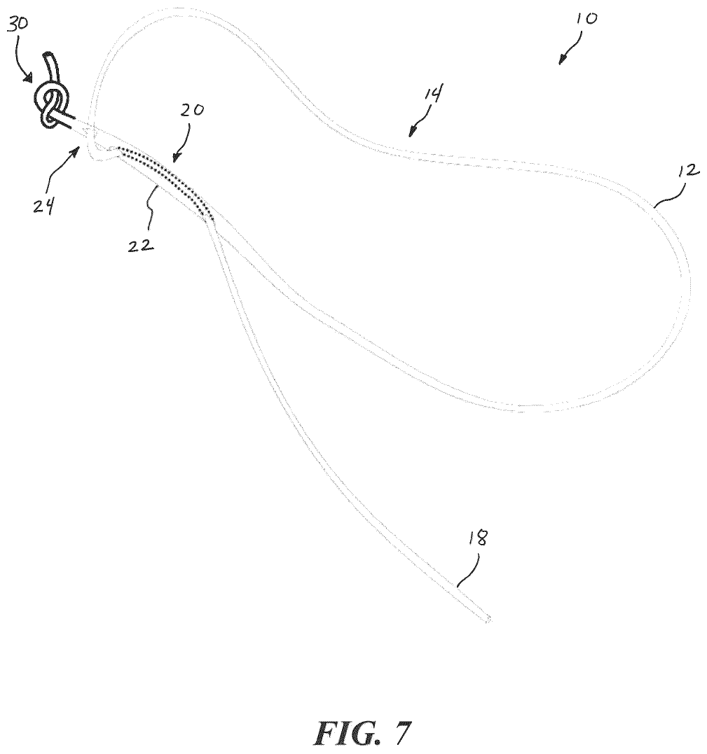

Referring to FIG. 7, in some embodiments, suture device 10 includes a fixed anchor 30 at the second end opposite the adjustable loop 14 instead of fixed loop 16. Fixed anchor 30 includes any suitable anchor such as but not limited to a knot tied in the suture body 12, flexible suture tape, button, suture material, metal anchor, PEEK anchor, plastic anchor, implant, graft, tissue graft, screw, plate, needle, suture loop, bioresorbable anchor, or other orthopedic devices fixed to or formed in the suture strand extending distally from the transverse pass 24. Fixed anchor 30 may be secured to an additional anchor, or may be positioned at the opening of an anchor, socket or tunnel, or may engage directly with tissue or bone to secure the end of the suture device 10 in a fixed position.

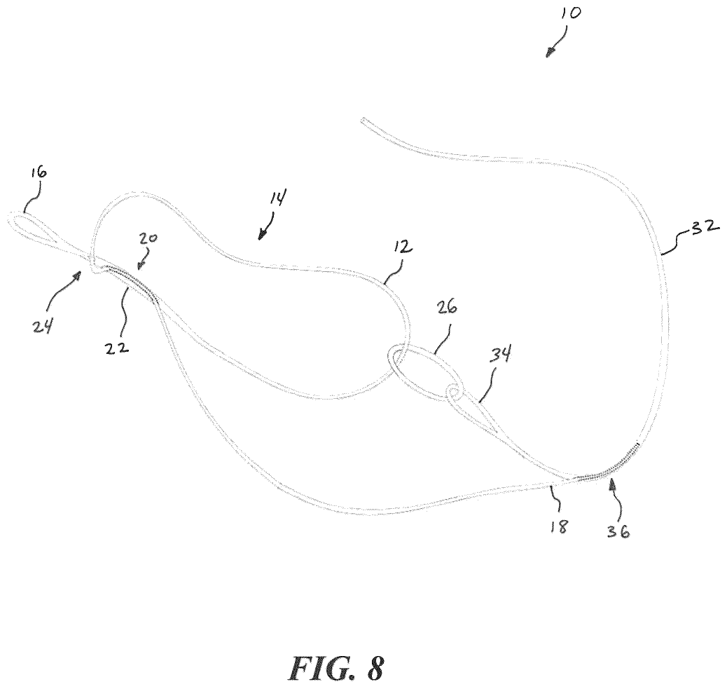

Referring to FIG. 8, in some embodiments, suture device 10 includes a first shuttling suture 32 attached to free loop 26. First shuttling suture 32 includes a first shuttling suture fixed loop 34 attached to free loop 26. First shuttling suture 32 may be used to pull suture device 10 through an anchor, bone or tissue, such as through a tunnel or socket drilled in bone. In some embodiments, first shuttling suture 32 includes a shuttling suture sleeve 36 positioned to receive a portion of tag end 18. In some embodiments, tag end 18 is inserted axially into the hollow core portion of first shuttling suture 32. As such, tag end 18 is protected as the suture device is pulled through an anchor, bone or tissue to a desired position. Once in position, tag end 18 may be removed from shuttling suture sleeve 36 and pulled to tighten adjustable loop 14.

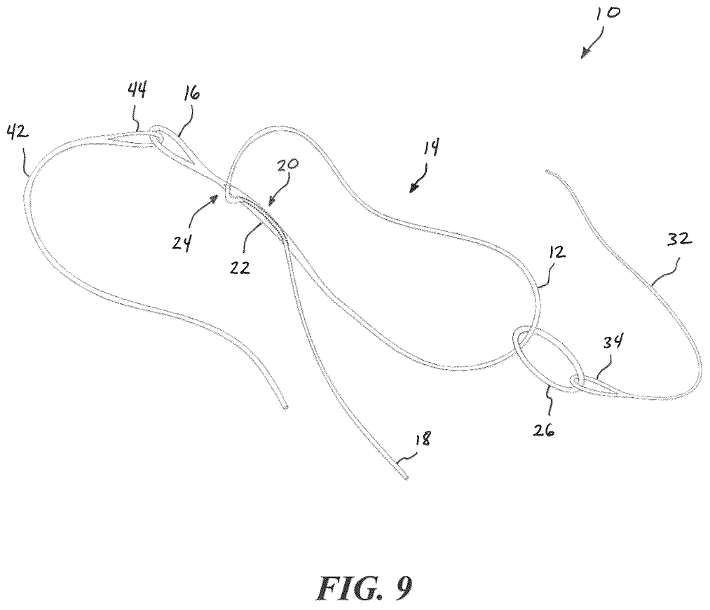

Referring to FIG. 9, in some embodiments, suture device 10 further includes a second shuttling suture 42 secured to fixed loop 16 via second shuttling suture fixed loop 44. Second shuttling suture 42 may be used to pull suture device 10 through an anchor, bone or tissue, such as through a tunnel or socket drilled in bone.

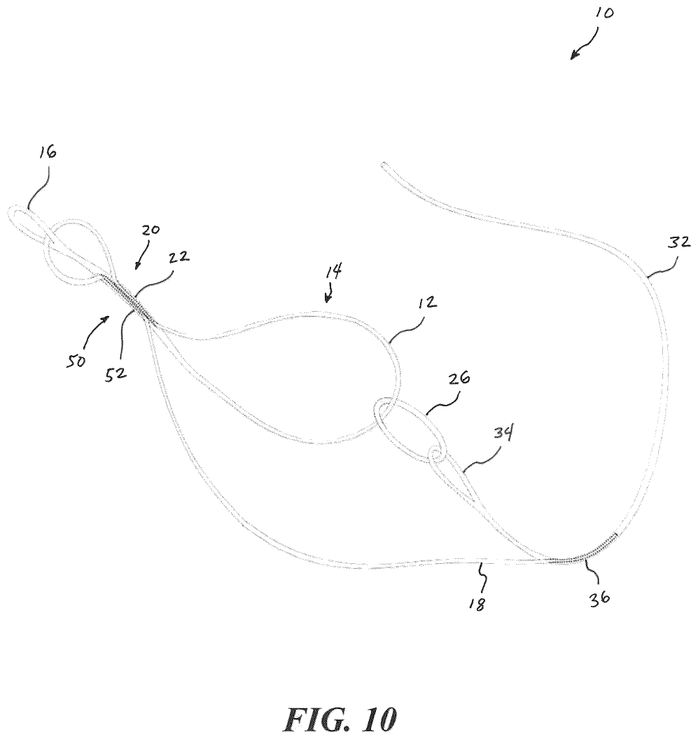

In further embodiments, suture device 10 includes dual self-cinching sections to provide enhanced self-cinching action of the suture construct when tensioned. Referring to FIG. 10, in some embodiments, suture device 10 includes a suture body 12 including a single strand of suture material forming an adjustable loop 14. The suture approaches fixed loop 16 and forms a first self-cinching section 20 wherein the suture strand passes axially through the hollow core of the suture material toward fixed loop 16. The suture strand then exits the hollow core and passes through fixed loop 16, makes a 180-degree turn, and passes toward adjustable loop 14. The suture strand then axially re-enters the hollow core of the suture material, forming a second self-cinching section 50 at a second self-cinching sleeve 52. The tag end 18 then travels toward adjustable loop 14 and may enter a shuttling suture sleeve 36 in shuttling suture 32.

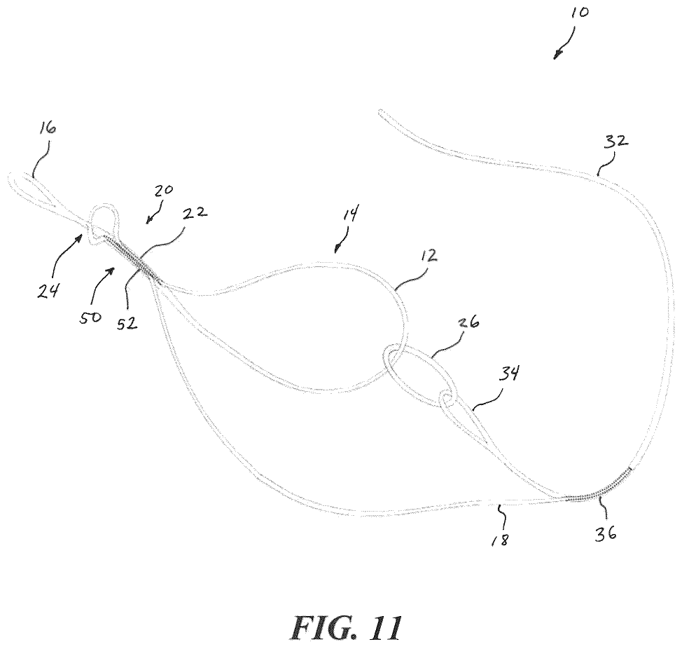

Referring to FIG. 11, in some embodiments, suture device 10 includes a similar configuration as shown in FIG. 10, but the suture strand passes transversely through the suture strand between first and second self-cinching sections 20, 50 and fixed loop 16 at a transverse pass 24 (instead of passing through fixed loop 16 as shown in FIG. 10).

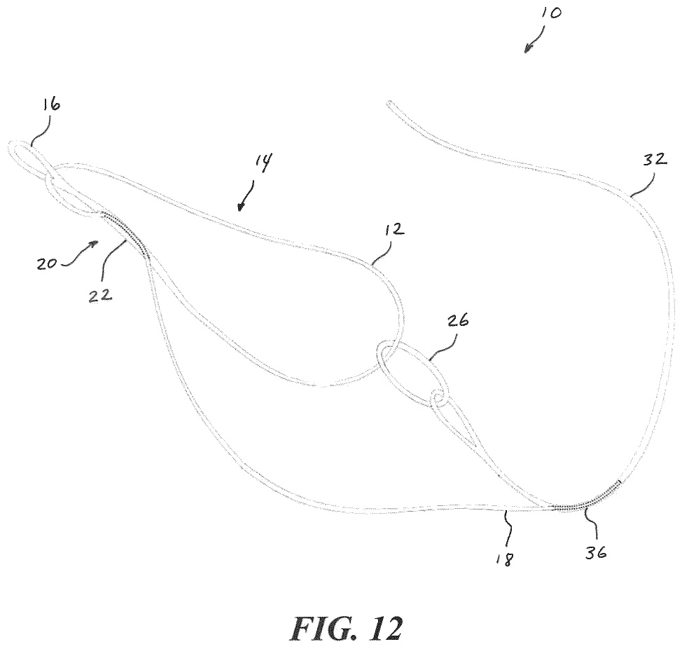

Referring to FIG. 12, in some embodiments, suture device 10 includes a configuration similar to the embodiment shown in FIG. 8, but the suture strand passes through fixed loop 16 (instead of passing transversely through the suture strand as shown in FIG. 8).

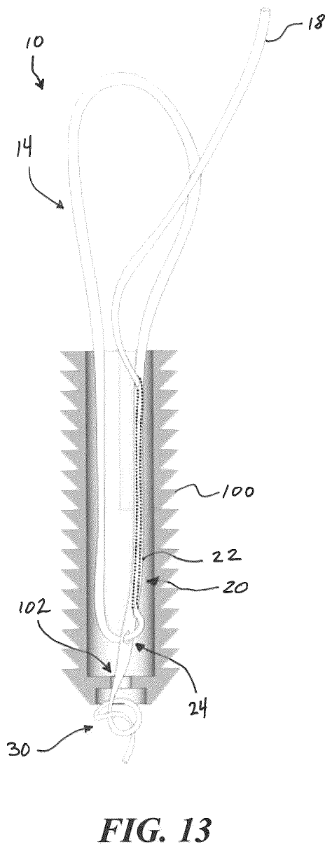

Referring to FIG. 13, an embodiment of a suture device 10 installed in a knotless suture anchor configuration is shown. An anchor 100 may include a threaded anchor installed in bone. The suture device 10 may be deployed into the anchor 100 by inserting the suture device 10 through the cylindrical bore of the anchor such that the distal end extends through a hole 102 at the distal end of the anchor 100. The suture may be tied into a knot outside of the bore to secure an end of the suture device and prevent the end of the suture device from sliding back into the bore due to the increased dimension of the knot being greater than the relevant dimension of the hole. From this position, the tag end 18 may be pulled to tighten adjustable loop 14. As tag end 18 is pulled, the suture strand slides through transverse pass 24 and through self-cinching section 20 including self-cinching sleeve 22. In some embodiments, the suture device 10 is attached to anchor 100 prior to insertion of the anchor 100 into the bone. In other embodiments, suture device 10 is attached to anchor 100 after anchor 100 is inserted into the bone.

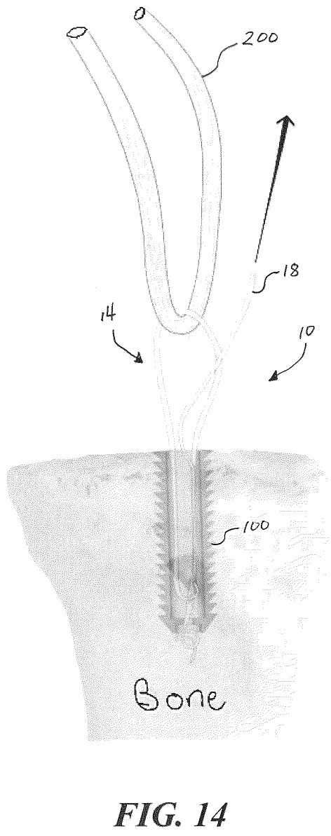

Referring to FIG. 14, a ligament or graft 200 may be installed in adjustable loop 14. As tag end 18 is pulled, adjustable loop 14 constricts, thereby pulling ligament or graft 200 down into the socket defined in anchor 100 adjacent the bone into a desired position for repair (with existing damaged ligament) or reconstruction (with graft).

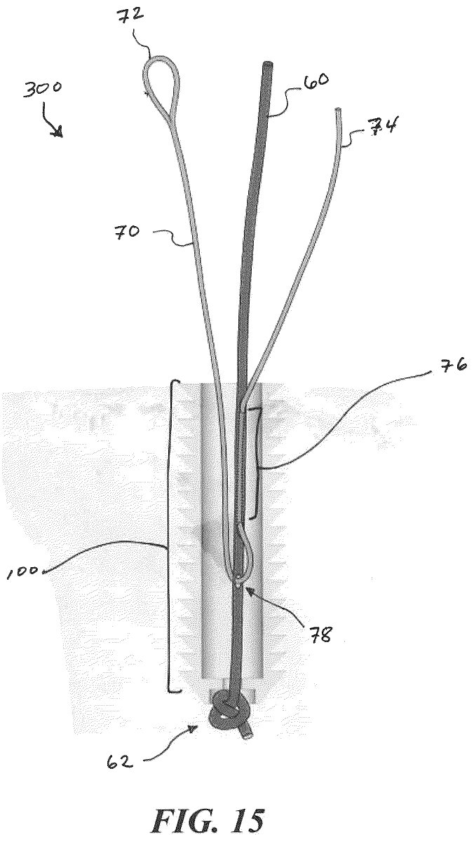

Referring to FIG. 15, an alternative embodiment of a suture construct 300 includes a main body suture 60 and a shuttling suture 70 installed on the main body suture 60 form a self-cinching suture construct in accordance with the present disclosure. The main body suture 60 of suture construct 300 is installed in an anchor 100 with a suture anchor 62 at the distal end. A shuttling suture 70 includes a shuttling suture fixed loop 72 and a suture strand extending down into the anchor, passing transversely through the main body suture 60 at a transverse pass 78, making a 180 degree turn, extending through a self-cinching section 76 in main body suture 60 and out of the anchor 100 to a tag end 74. From this position, the shuttling suture 70 may be used to affix a ligament or a graft to the main body suture 60 in a self-cinching device configuration for repair of torn tissue such as but not limited to a labrum, rotator cuff or other ligaments and tendons.

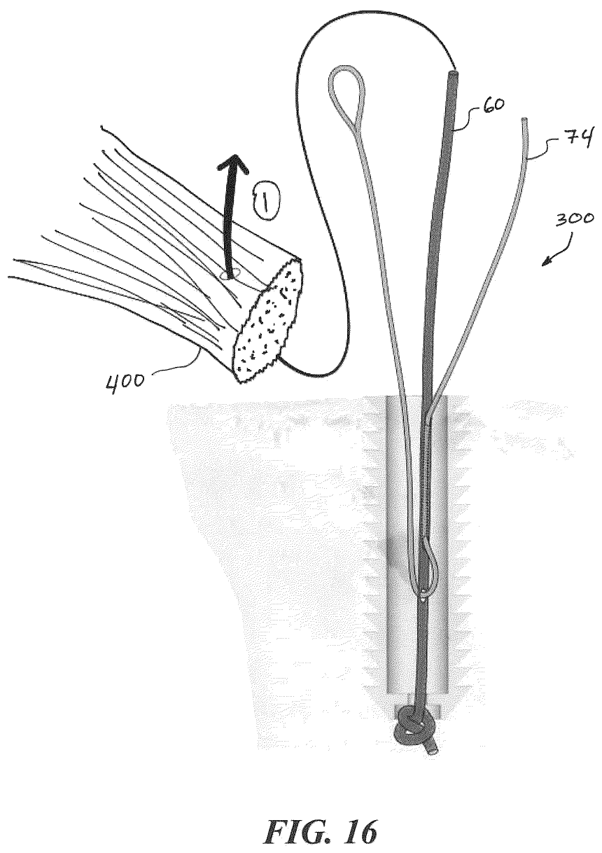

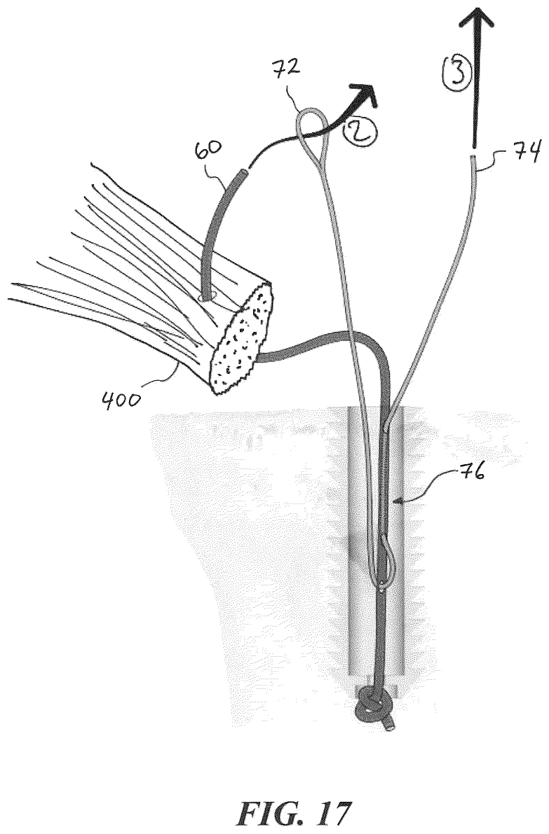

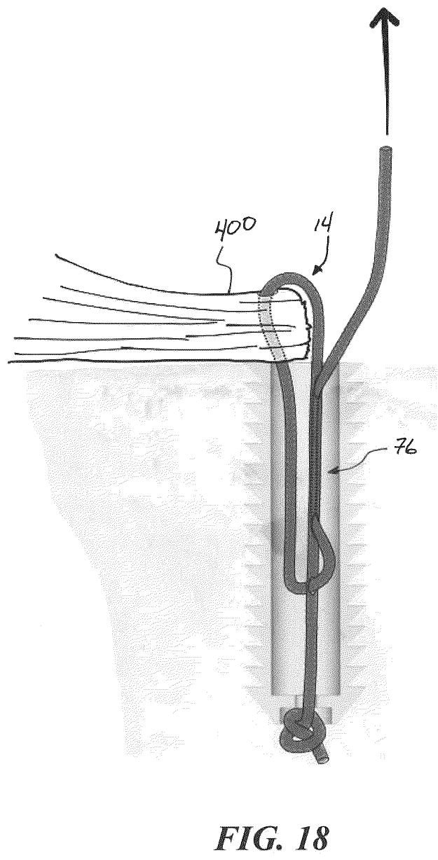

Referring to FIG. 16, in an example of use of the suture construct 300, main body suture 60 is installed through a ligament or tendon to be repaired 400. Next, referring to FIG. 17, main body suture 60 may be passed through suture fixed loop 72 after passing through the ligament or tendon to be repaired 400. From this position, the tag end 74 may be pulled to draw suture fixed loop 72 down into the anchor, thereby pulling main body suture 60 and through itself at transverse pass 78, and through itself at self-cinching section 76, resulting in a configuration as shown in FIG. 18 with ligament or tendon 400 approximated against the bone in a desired position and forming adjustable loop 14.

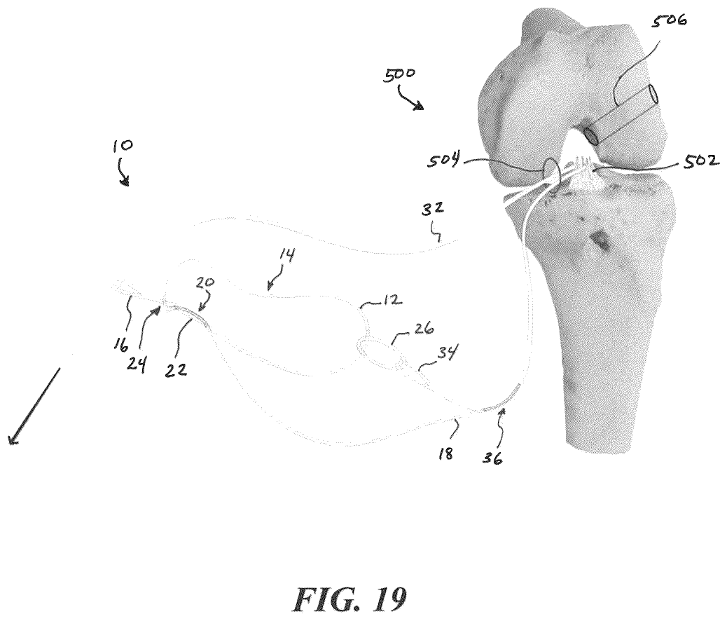

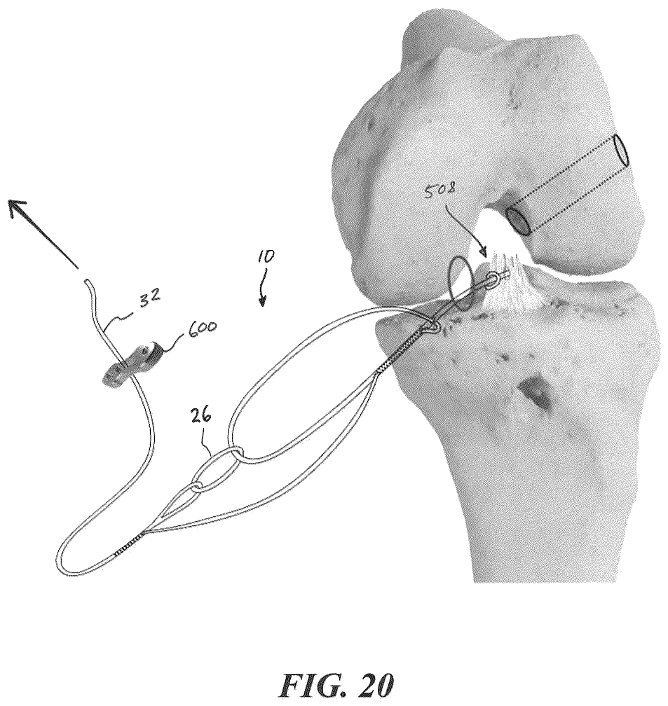

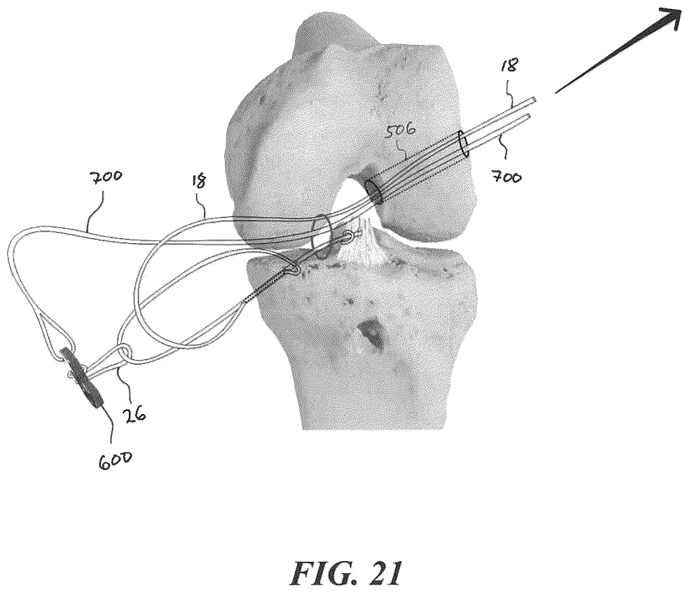

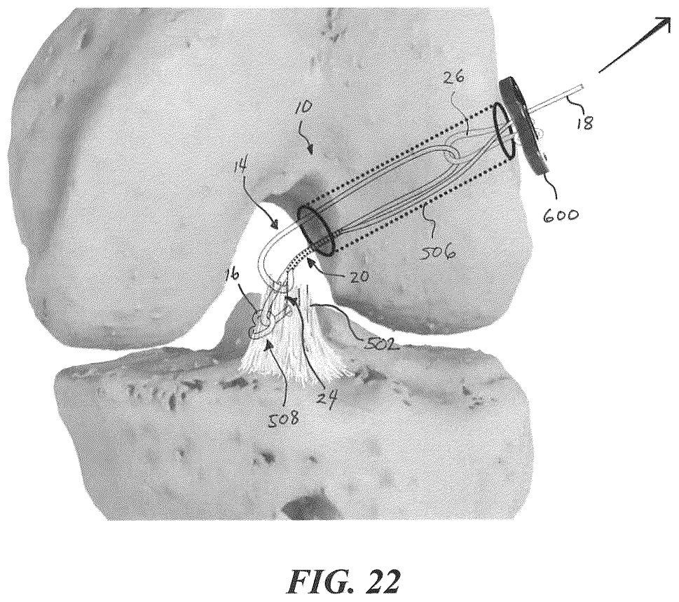

Referring to FIGS. 19-22, an example of a method of transosseous tissue repair utilizing a suture device of the present disclosure is illustrated. Referring to FIG. 19, an embodiment of a suture device 10 (as shown previously in FIG. 8) is positioned for performing surgery on a knee 500 to repair a torn ACL tendon 502. Shuttling suture 32 is inserted through an incision 504 and passed through the tendon 502. The shuttling suture 32 is passed through fixed loop 16. The shuttling suture 32 is then pulled, thereby pulling the entire suture construct through the ACL tendon 502 and forming a hitch with fixed loop 16 on the tendon 502 and orienting the suture device 10 for further installation, as shown in FIG. 20. At this position, an anchor such as a suture button 600 is installed onto the shuttling suture 32 and advanced toward free loop 26. Suture button 600 is then secured to free loop 26 by hitching free loop 26 around suture button 600, as shown in FIG. 21.

Referring further from FIG. 21, from this position, a passing suture 700 installed on suture button 600 is advanced through tunnel 506, and also tag end 18 is advanced through tunnel 506. By pulling on passing suture 700, suture button 600 is pulled into the knee and through tunnel 506 to the femoral cortex on the outside of the tunnel 506. Suture button 600, once outside of tunnel 506, the suture button 600 forms an anchor outside of the tunnel. In this position, suture device 10 is deployed in position for completing the ACL repair procedure. From this position, the fixed loop 16 is formed in a hitch 508 around the tendon 502. Tag end 18 may be pulled, which draws the suture strand through self-cinching section 20 and transverse pass 24, thereby tightening adjustable loop 14. As such, tendon 502 is pulled upwardly toward the tunnel 506 into a desired position. Once tightened, the self-cinching section 20 and transverse pass 24 work cooperatively to maintain tension on the suture device 10 to hold the tendon 502 into a desired position, while the hitch 508 tightens against the tendon 502. Once the desired tension is applied, tag end 18 may be trimmed or tied into a knot on the exterior of suture button 600.

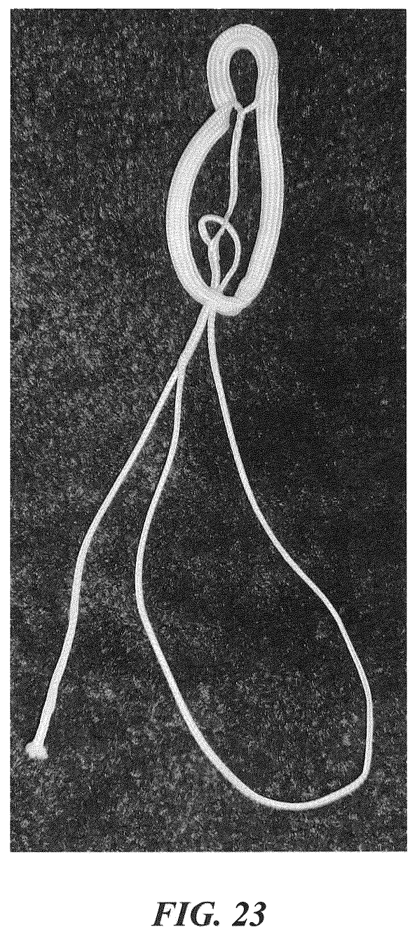

Referring to FIG. 23, an alternative embodiment of a self-cinching suture device is shown including a first anchor forming a tubular, hollow-core anchor in a ring shape. The Suture devices passes transversely through one end of the anchor at the self-cinching section and the adjustable loop, and the fixed loop is installed axially through the interior of the hollow core of the anchor. During use, the anchor may slide toward the fixed loop and gather together to form an occlusion outside of a tunnel or socket.

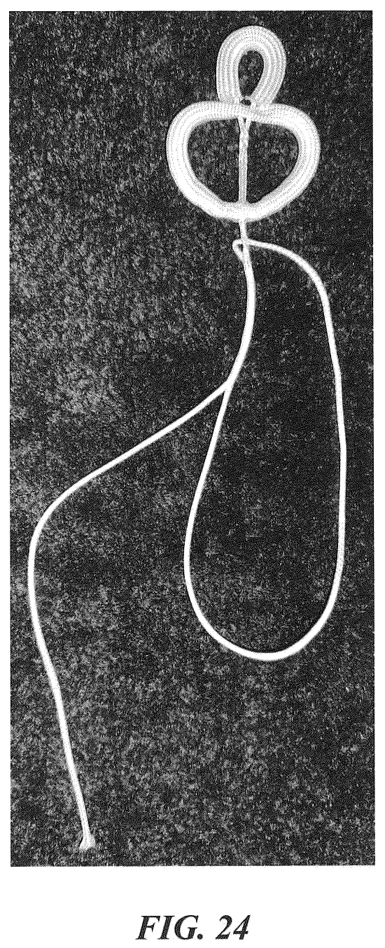

Referring to FIG. 24, in an alternative embodiment, a self-cinching suture device includes a first anchor disposed on the fixed loop and a second anchor disposed on the suture strand between the transverse pass and the first anchor. The first anchor includes a tubular sleeve, with the fixed loop disposed axially inside the tubular sleeve. The second anchor includes a second tubular sleeve formed in a ring shape, wherein the suture strand passes transversely through the second anchor twice. During use, the second anchor slides along the suture strand toward the first anchor and forms, and the first anchor compresses around the fixed loop to form an occlusion outside of a tunnel or socket.

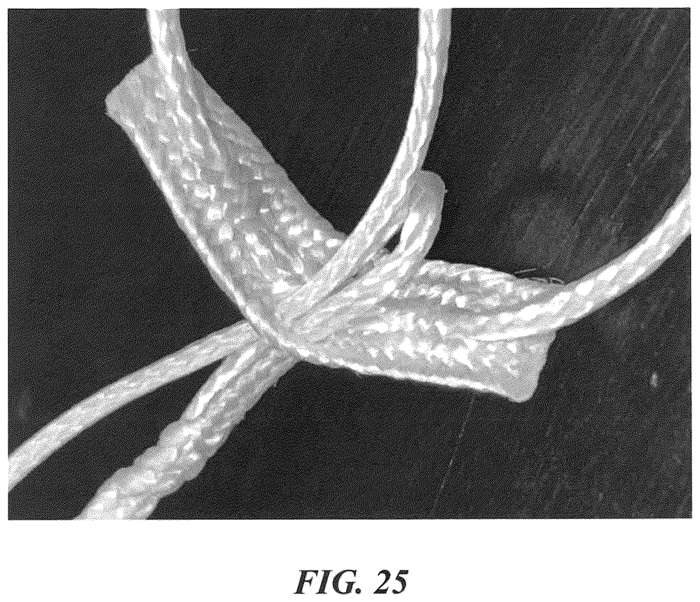

Referring to FIG. 25, an alternative embodiment of an anchor disposed on an adjustable loop is shown. The anchor comprises flexible suture tape or flat suture material. The suture strand passes through the suture tape in one or more passes along the length of the anchor. Similarly the tag end of the suture body passes transversely through the anchor, and the fixed loop of a shuttling suture also passes transversely through the anchor.

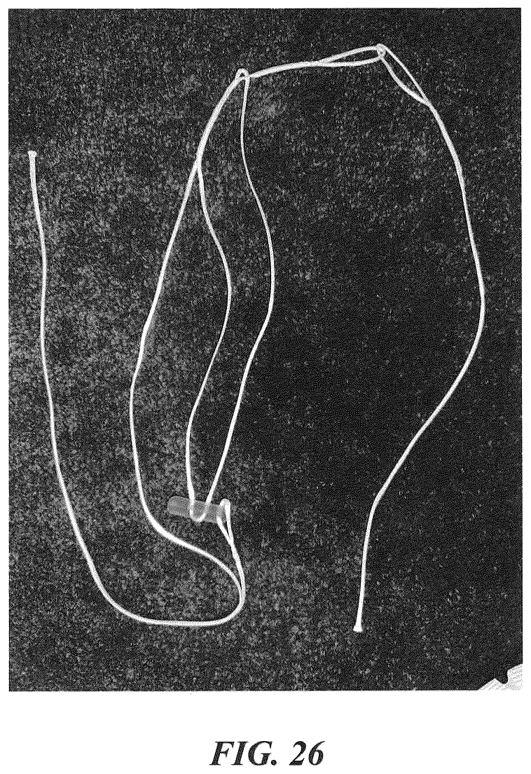

Referring to FIG. 26, an alternative embodiment of a self-cinching suture device is shown with a pre-loaded suture button on the adjustable loop and a passing suture disposed on an end of the suture button to facilitate the method disclosed in FIGS. 19-22.

What is claimed is:

1. A self-cinching suture apparatus, comprising:
a suture body including a fixed loop and an adjustable loop opposite the fixed loop;
a self-cinching section defined in the suture body wherein a portion of the suture body passes axially through the suture body, forming a tag end extending from the self-cinching section in a direction away from the fixed loop;
a transverse pass between the self-cinching section and the fixed loop, wherein a portion of the suture body passes transversely through the suture body at the transverse pass.

2. The apparatus of claim 1, further comprising a free loop disposed on the adjustable loop.

3. The apparatus of claim 2, further comprising a first anchor disposed on the free loop.

4. The apparatus of claim 3, wherein the first anchor comprises a soft anchor.

5. The apparatus of claim 2, further comprising a first shuttling suture disposed on the free loop.

6. The apparatus of claim 5, further comprising a second shuttling suture disposed on the fixed loop.

7. The apparatus of claim 3, further comprising a second anchor disposed on the fixed loop.

8. The apparatus of claim 7, wherein the second anchor comprises a soft anchor.
